(19) United States
(12) Patent Application Publication
(10) Pub. No.: US 2008/0183180 A1
(43) Pub. Date: Jul. 31, 2008

(54) Implant Holder and Pusher

(75) Inventors: Richard W. Franks, Memphis, TN (US); Harold S. Taylor, Memphis, TN (US); Rolando Puno, Prospect, KY (US); George Frey, Englewood, CO (US)

Correspondence Address: HAYNES AND BOONE, LLP, 901 Main Street, Suite 3100, Dallas, TX, 75202, US

(73) Assignee: Warsaw Orthopedic, Inc., Warsaw, IN (US)

(21) Appl. No.: 11/669,818

(22) Filed: Jan. 31, 2007

(51) Int. Cl. A61B 17/58

(52) U.S. Cl. 606/99

(57) ABSTRACT

An implant holder and driver for use in implanting orthopedic implants is disclosed. The instrument includes a distal retaining member that affirmatively holds the orthopedic implant and allows significant force to be applied by the driver without dislodging the engagement. An actuation mechanism is provided to move the retaining member between the open and closed positions. The actuation mechanism utilized in one aspect of the present invention may be operated in an ergonomic and efficient fashion.

### FIELD OF THE INVENTION

The present invention relates generally to instruments used to manipulate orthopedic implants. More particularly, the present invention relates to the manipulation of bone anchoring elements positioned in the spine that may be connected to longitudinal members extending along the length of the spine. While the invention is particularly useful in spinal surgery, it may find use in other areas of medicine as well.

### BACKGROUND OF THE INVENTION

In many applications, particularly those related to spinal correction techniques, it is desirable to place a series of implants in a patient's spine prior to inserting a longitudinal member (a rod or a plate) along the spine to interconnect the previously placed implants. Placement of the bone anchoring elements can be a challenging surgical procedure as the surgical access to the spinal column is limited and there is a need for very precise placement.

As a result of the demands of the surgical procedure, there remains a need for improvements to surgical instruments that can hold and push orthopedic implants.

### SUMMARY OF THE INVENTION

In one aspect of the present invention, there is provided a surgical instrument for placement of orthopedic implants. The surgical instrument comprises a shaft having a distal portion, an opposite proximal portion and a longitudinal axis. The surgical instrument includes a driving surface associated with an implant receiving area extending distally from the shoulder. A retaining member extends distally from the implant receiving area and is adapted to be moveable to hold an implant to the surgical instrument.

In another aspect of the present invention, there is provided a combination orthopedic implant and surgical instrument for holding and driving the implant. This combination comprises a surgical instrument with a driving area and retaining member operable on the distal portion of the implant between a locked retaining position and a released open position.

In yet a further aspect of the present invention, there is provided a method for implanting an orthopedic implant. The surgical method comprises providing a surgical instrument having a driving surface and a distally extending retaining member. An implant is positioned adjacent the driving surface and the retaining member is moved to engage the implant and retain it adjacent the driving surface. The implant is positioned in a patient's body, the retaining member is moved to a spaced relation away from the implant, and the surgical instrument is removed.

Related objects and advantages of the present invention will be apparent from the following description.

### BRIEF DESCRIPTION OF THE DRAWINGS

FIG. 1 is a perspective view of a surgical instrument in accordance with one aspect of the present invention.

FIG. 2 is an enlarged partial perspective view of the implant driver portion of the surgical instrument of FIG. 1.

FIG. 3 is an enlarged partial cross-sectional view of the actuation mechanism of the surgical instrument of FIG. 1.

FIG. 4 is a perspective view of a portion of the surgical instrument of FIG. 1 engaged with an orthopedic implant.

FIG. 5 is a top view of the surgical instrument of FIG. 4.

FIG. 6 is a side view of the surgical instrument of FIG. 4 and the orthopedic implant being implanted in a spinal segment.

### DESCRIPTION OF THE PREFERRED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such applications of the principles of the invention as illustrated herein being contemplated as would normally occur to one skilled in the art to which the invention relates.

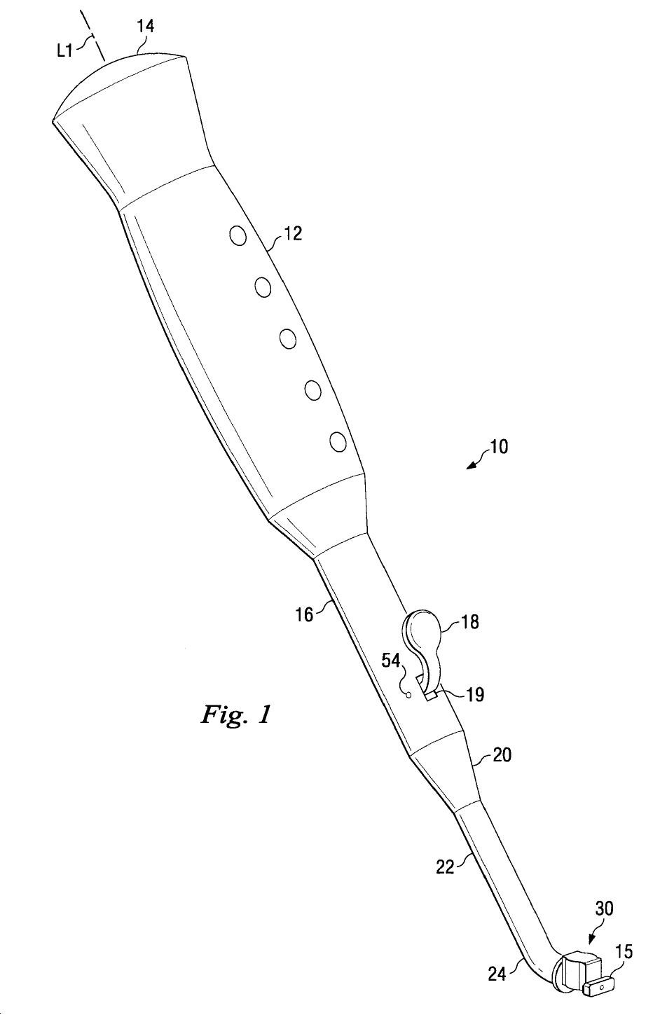

Referring now to FIG. 1, an implant holder 10 according to the present invention is illustrated. Implant holder 10 includes a handle 12 adjacent to the proximal end 14. Extending towards distal end 15 and away from the handle 12, implant holder 10 narrows to shaft 16 which includes a thumb actuator 18 extending out of aperture 19. The shaft includes taper 20 extending to distal, hollow shaft 22 which includes a bend 24 and an implant driver assembly 30. Implant holder 10 includes a longitudinal axis L1 extending along a substantial portion of handle 12 and shaft 16. It will be appreciated that implant driver assembly 30 extends at an oblique angle with respect to the longitudinal axis L1.

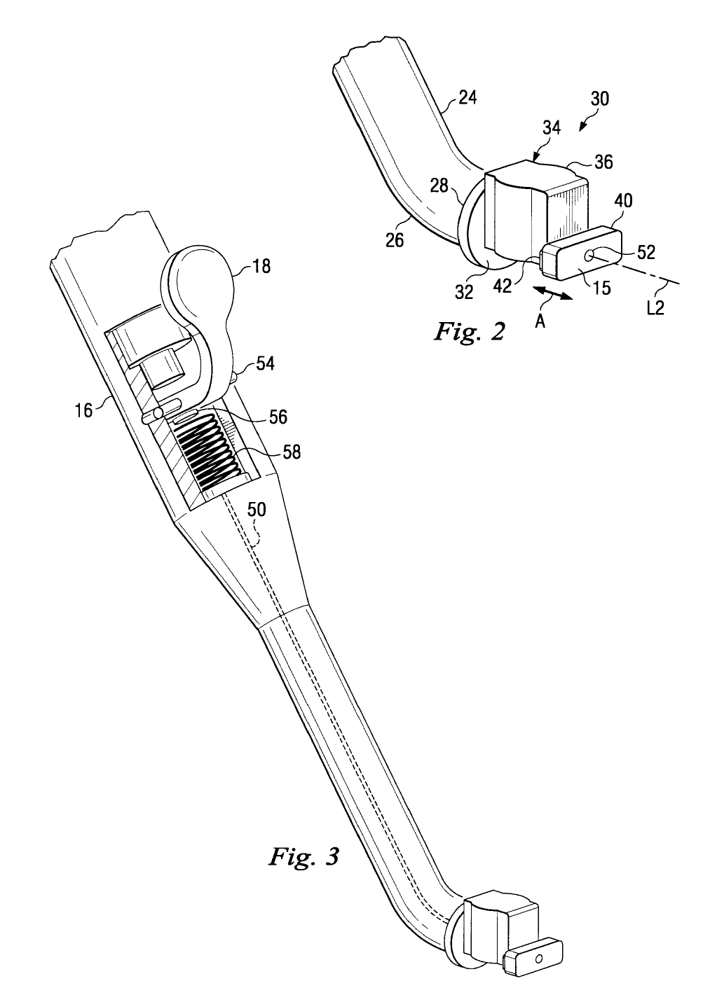

Referring now to FIG. 2, there is shown an enlarged view of the implant driver assembly 30 of FIG. 1. Implant driver assembly 30 includes an enlarged flange 28 having a distal implant driving surface 32. Continuing distally toward distal end 15, driving assembly 30 includes implant receiving area 34 which has a pair of intermediate protrusions 36 extending outwardly from the longitudinal axis L2 that extends along a substantial portion of implant driving assembly 30. At the distal most portion of driving assembly 30, the device includes a retaining member 40. Retaining member 40 has a pair of proximal projections 42 and 44 (see also FIG. 5). Additionally, retaining member 40 includes an implant engaging surface disposed on its proximal portion configured and adapted for engaging an orthopedic implant. Retaining member 40 is attached to driving assembly 30 via a cable 50 (see FIG. 3) that engages retaining member 40 at attachment 52. Retaining member 40 is configured and adapted to be moveable in the direction of arrow A proximally and distally along longitudinal axis L2. Retaining member 40 can be moved distally along longitudinal axis L2 into a released position, as shown in FIG. 2, adapted for receiving an implant or releasing a previously engaged implant. Similarly, movement of retaining member 40 proximally along longitudinal axis L2 moves the retaining member 40 to a retaining position, as shown in FIG. 6, for holding an implant on the implant receiving area 34.

Referring now to FIG. 3, there is shown an enlarged partial cross-sectional view of the actuation mechanism for implant holder 10 and the associated implant driver assembly 30. The actuation mechanism includes a thumb actuator 18 joined to shaft 16 via pivot pin 54 to create a freely moveable actuator arm 56 opposite thumb actuator 18. Actuator arm 56 is joined to cable 50 (shown in dashed lines) which extends along the length of the device inside shaft 22 between the actuator 18 and retaining member 40. In one aspect of the present invention, a biasing member such as spring 58 is disposed about cable 50 and engages actuator arm 56 to urge the actuation mechanism into the locked position. It will be appreciated that spring 58 applies a force on cable 50 that has a tendency to pull retaining member 40 proximally towards driving surface 32.

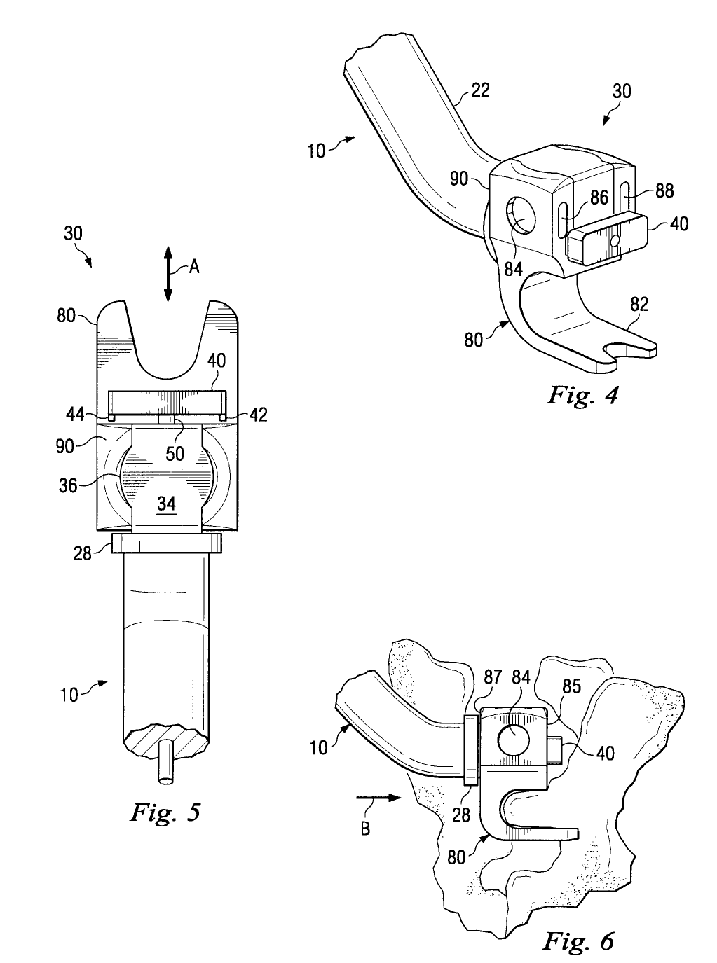

Referring now to FIGS. 4-6, the implant holder 10 will now be described in use with an orthopedic implant. Specifically, implant holder 10 will be disclosed and described for use with placement of a spinal hook 80. Spinal hook 80 includes a bone engagement area 82 and external surface 84. Disposed within the external surface along its distal end are a pair of receiving recesses 86 and 88. Further, as known in the art, hook 80 has an internal cavity 90 adapted to receive a longitudinal spinal rod and a retaining member (not shown) to join hook 80 to the rod.

In use, driving assembly 30 is positioned and aligned above internal cavity 90 with the implant mating area 34 positioned such that protrusions 36 substantially match and align with the internal contours of cavity 90. The implant is then moved transverse to longitudinal axis L2 to seat implant mating area 34 within internal cavity 90. In the illustrated embodiment, this movement is conducted with one hand positioned on handle 12 and the thumb of the same hand depressing actuator 18 such that retaining member 40 is in the released position. As will be understood, it is possible that the operator may use the opposing hand to hold the spinal implant before insertion into the implant holder 10. In the alternative, spinal hook 80 may be positioned in a hook holder or otherwise in a tray such that it may be picked up by implant holder 10 using only the single hand positioned on the instrument. It will be appreciated that in one aspect, although not required for all aspects, the present invention provides an instrument for gripping and driving an implant that is operable for these functions with only one hand of the user. After implant mating area 34 is positioned within internal cavity 90 the operator may release thumb actuator 18. Biasing member 58 will then apply tension on cable 50 to urge retaining member 40 against the exterior surface 84 of the implant. In the illustrated embodiment, the proximal surface of retaining member 40 includes projections 42 and 44 that may be received within receiving cavities 86 and 88 on the implant, respectively. It will be appreciated that while projections 42 and 44 are disposed within recesses 86 and 88, respectively, the implant 80 cannot be removed from the implant mating area 34.

In one aspect, implant mating area 34 is sized and configured to have a slightly smaller external dimension than the internal dimension of internal cavity 90 to permit the implant to move proximally in response to force applied by the retaining member 40 on the distal exterior surface of the implant. In this manner, the retaining member 40 urges the proximal outer surface 87 on the proximal portion of hook 80 against driving surface 32 of flange 28. As a result, implant 80 is squeezed between the implant engaging surface of the retaining member 40 and the driving surface 32. Further, when it is necessary to apply a driving force on implant 80 the force is transmitted from driving surface 32 to the exterior surface 87 of the implant. In the illustrated aspect, implant receiving area 34 and retaining member 40 can be formed in a less robust form permitting a lower profile since they do not need to be constructed to transmit a driving force to the implant 80. Moreover, the protrusions 36 on implant mating area 34 inhibit rotation of implant 80 with respect to implant holder 10. As a result, the hook 80 is firmly attached to implant holder 10 and may be driven with significant force into position.

In an alternative configuration, the flange 28 and associated driving surface 32 may be greatly diminished or omitted. In this configuration, implant receiving area 34 is configured with a driving surface to engage the interior surface of the cavity of the implant. It will be understood that at least a portion of the implant receiving area 34 will be configured for engagement with the implant to transmit impaction forces to the implant.

As will be appreciated from FIG. 5, retaining member 40 has a constant width from the lateral side adjacent projection 42 to the opposite lateral side adjacent projection 44 regardless of whether it is positioned in the retaining position or the releasing position. In the illustrated embodiment, the width of the retaining member 40 is less than the width of the implant 80. Further, the distal most portion of the retaining member 40 (see FIG. 6) remains proximal of the greatest distal projection of implant 80 when held by the implant holder 10. As such, the retaining member 40 can be moved between the retaining position and the releasing position without increasing the total length or width of the combination of the implant holder 10 and the implant 80.

As shown in FIG. 6, hook 80 has been positioned adjacent the lamina of a spinal segment. If needed, a mallet (not shown) can be used to impact on proximal portion 14 adjacent handle 12 to drive the hook into position and firmly engage it in the spine. Further use and description of spinal hooks is not described herein, however U.S. Pat. Nos. 5,910,141 and 5,246,442 are incorporated by reference herein in their entirety.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

What is claimed is:

1. A surgical instrument for placement of orthopedic implants having an interior cavity and an exterior surface, the surgical instrument comprising:
a shaft having a distal portion, an opposite proximal portion and a longitudinal axis;
a driving shoulder disposed adjacent said distal portion and an implant receiving area extending distally from said driving shoulder; and
a retaining member extending distally from said implant receiving area, said retaining member having a first retaining position configured for retaining an implant adjacent said implant receiving area and a second released position for allowing removal of an implant from said implant receiving area.

2. The surgical instrument of claim 1, wherein said retaining member is biased toward said first retaining position.

3. The surgical instrument of claim 2, further including a biasing member disposed within said shaft and connected to said retaining member.

4. The surgical instrument of claim 3, further including an actuator disposed between said distal portion and said proximal portion, said actuator movable to overcome said biasing member and urge said retaining member to said second released position.

5. The surgical instrument of claim 4, wherein said actuator is moved linearly to control said retaining member between said first retaining position and said second released position.

6. The surgical instrument of claim 5, wherein said actuator is moved transverse to said longitudinal axis.

7. The surgical instrument of claim 1, wherein said retaining member in said retaining position urges the implant proximally against said driving shoulder.

8. The surgical instrument of 3, further including a control member interconnected between said actuator and said retaining member, said control member extending along said shaft of the surgical instrument.

9. The surgical instrument of claim 8, wherein said biasing member is a helical spring disposed about a portion of said control member.

10. The surgical instrument of claim 6, wherein said actuator is a thumb actuator positioned on along the shaft to permit the user to hold the shaft for stability and actuate said thumb actuator with a single hand.

11. The surgical instrument of claim 1, wherein said implant receiving area is configured for placement at least in part within the interior cavity of the implant, said implant receiving area having a second longitudinal axis.

12. The surgical instrument of claim 11, wherein implant receiving area cooperates with the interior cavity of the implant to inhibit rotational movement of the implant with respect to the second longitudinal axis.

13. The surgical instrument of claim 1, wherein the retaining member has at least one proximal projection extending toward the implant

14. The surgical instrument of claim 13, wherein the implant has at least one retention cavity on the exterior surface and said projection is configured for placement within said retention cavity.

15. The surgical instrument of claim 11, wherein said driving shoulder is oriented substantially perpendicular to said second longitudinal axis, and the second longitudinal axis extends obliquely with respect to the longitudinal axis.

16. The surgical instrument of claim 4, wherein said actuator moves said retaining member along said second longitudinal axis.

17. The surgical instrument of claim 1, wherein said distal portion has a first width and said retaining member is configured for movement between said first retaining position and said second released position without increasing the first width of the distal portion of the surgical instrument.

18. The surgical instrument of claim 8, wherein said control member is flexible.

19. The surgical instrument of claim 18, wherein said control member is a cable.

20. A combination orthopedic implant and surgical instrument, the combination comprising:
a orthopedic implant having an interior cavity and an exterior surface, and a bone engaging surface formed on said exterior surface; and
a surgical instrument having a shaft, a distal portion, an opposite proximal portion and a longitudinal axis, a driving shoulder disposed adjacent said distal portion and an implant mating area configured for at least partially positioning within said interior cavity and extending distally from said shoulder; and a retaining member extending distally from said implant mating area, said retaining member having a first retaining position configured for retaining an implant proximal said implant mating area and a second released position for allowing removal of an implant from said implant mating area.

21. The combination of claim 20, wherein said orthopedic implant is a spinal implant.

22. The combination of claim 21, wherein said orthopedic implant is one of a plurality of implants formed with different external sizes and having similar internal cavities for instrument engagement surfaces.

23. The combination of claim 22, wherein said spinal implant is a hook.

24. A method for implanting an orthopedic implant, comprising:
providing a surgical instrument having a shaft, a distal portion, an opposite proximal portion and a longitudinal axis, a driving shoulder disposed adjacent said distal portion and an implant receiving area extending distally from said shoulder, and a retaining member extending distally from said implant receiving area;
positioning the implant receiving area within a recess of an orthopedic implant and adjacent the driving shoulder;
moving the retaining member proximally to engage a distal portion of the orthopedic implant;
implanting the orthopedic implant in a patient;
moving the retaining member distally to a position spaced from the orthopedic implant; and
releasing the orthopedic implant from the surgical instrument.

25. The method of claim 24, wherein said moving the retaining member proximally further includes urging a proximal portion of said implant against the driving shoulder.

26. The method of claim 24, wherein the surgical instrument includes an actuator and said moving the retaining member proximally is performed by moving the actuator to a locked position and said moving the retaining member distally is performed by moving the actuator to a released position.

27. The method of claim 25, wherein the surgical instrument includes a biasing member and said moving the actuator to a locked position is performed by the force of the biasing member.

28. A surgical instrument for placement of orthopedic implants having an interior cavity and an exterior surface, the surgical instrument comprising:
a shaft having a distal portion, an opposite proximal portion and a longitudinal axis;
an implant receiving area disposed adjacent said distal portion, said implant receiving area including at least one driving surface, and
a retaining member extending distally from said implant receiving area, said retaining member having a first retaining position configured for retaining an implant adjacent said implant receiving area and a second released position for allowing removal of an implant from said implant receiving area.

29. The surgical instrument of claim 28, wherein said at least one driving surface is disposed on an exterior surface of said implant receiving area and is configured for engaging a portion of the interior cavity of the implant.

30. The surgical instrument of claim 28, wherein said at least on driving surface is disposed on the proximal portion of said implant receiving area and is configured for engaging a portion of the exterior surface of the implant.
